(19) United States
(12) Patent Application Publication
(10) Pub. No.: US 2016/0095570 A1
(43) Pub. Date: Apr. 7, 2016

(54) Method for detecting teeth grinding

(71) Applicant: Michiel Allessie, Broek in Waterland (NL)

(72) Inventor: Michiel Allessie, Broek in Waterland (NL)

(21) Appl. No.: 14/504,452

(22) Filed: Oct. 2, 2014

(51) Int. Cl. A61B 7/00; A61B 5/00; A61B 5/083; A61B 5/0205

(52) U.S. Cl. CPC A61B 7/006; A61B 5/02055; A61B 5/0077; A61B 5/0836; A61B 5/7282; A61B 5/024

(57) ABSTRACT

A method for detection of teeth grinding using audio which is recorded by a mobile device. The mobile device preferably comprises a mobile phone, a Personal Digital Assistant (PDA), a tablet computer, or a laptop. The audio that is recorded is analyzed by a computer program using pattern recognition and machine learning. The computer program may be trained to determine a patient's personal teeth grinding sounds. For this purpose a personal algorithm for each patient to detect teeth grinding sounds may be created.

### TECHNICAL FIELD

The present invention relates to systems and methods for detecting teeth grinding.

### BACKGROUND

The present invention relates to a system and method for the treatment of sleeping disorders such as teeth grinding and jaw clenching (hereinafter referred to as clenching). The term bruxism covers in this respect the abnormal excessive and non-functional nocturnal or subconscious grinding of teeth and clenching. Hereinafter the term bruxism is used to cover both teeth grinding and clenching whilst teeth grinding is referred to as a particular form of bruxism.

At a minimum, bruxism will typically result in excessive tooth wear and periodontal problems. Unfortunately in many cases this bruxing action not only damages the teeth themselves, but also the supporting structure of the teeth including both the hard bony material and the soft tissue. As a result, in more extreme cases these disorders lead to temporomandibular disorders, jaw displacement, stiff neck, and severe headaches.

Whenever the word patient is used hereinafter, it should be understood that this term implies any person, whether he or she is ill, suffering, in need for a treatment, hospitalized or is in none of these conditions. Whenever the word “his” or “he” is used, one may read also “her” or “she” respectively.

Bruxism is often classified as either awake bruxism or sleep bruxism. Awake bruxism is the condition wherein a patient habitually clenches his teeth and jaw when awake, usually without any teeth grinding. Most patients will do this subconsciously while concentrating. Sleep bruxism is the condition wherein a patient subconsciously grinds his teeth and squeezes his jaw muscles in his sleep. Partners may hear the grinding, which can be noisy.

Bruxism is then further divided into primary and secondary bruxism. Primary bruxism occurs without any underlying medical condition. Secondary bruxism is caused by another condition such as depression or anxiety, medication such as antidepressants, or the use of recreational drugs such as cocaine and ecstasy.

Stress and anxiety are thought to make sleep bruxism more likely, or worse. Bruxism is also more prevalent in people who regularly drink alcohol, smoke tobacco or drink caffeine (in particular when more than six cups a day). Although not everyone who bruxes is under stress, it has been shown that some people are bruxing more after a tense day, or in the anticipation of stress. As stress, or the perception of stress occurs, bruxism is likely to occur.

One traditional treatment for teeth grinding involves placing a protective layer of acrylic material, generally referred to as a splint, over the involved teeth. This acrylic layer serves to protect the teeth from direct contact, thus reducing wear, and to spread the load caused by clenching so that it is shared more equally among the neighboring teeth. This reduces wear of the teeth, but the acrylic protective layer does not address the problem of clenching, which frequently continues indefinitely. In some cases the urge to clench is worsened.

Since bruxism may often be the result of stress, typical traditional solutions which focus on guarding against the physical consequences of bruxing fail to permanently relieve the patient. A permanent solution may lie either in eliminating the stress causing agent or in improving stress management skills.

One method of treating bruxism is behavior modification. Behavior modification typically involves directing a stimulus, sometimes an unpleasant one, at the patient whenever he or she practices the undesirable behavior. Thus the patient gradually learns not to perform the undesirable behavior, thus avoiding the unpleasant stimulus.

U.S. Pat. No. 4,934,378 discloses a system for monitoring bruxism by measuring the electrical signals emitted by the jaw muscles. The monitoring apparatus is mounted on a probe that is inserted into one of the user's ear channels. When the system detects jaw muscle activity associated with bruxism, it alerts the user, for example by emitting an audible tone or a prerecorded message.

U.S. Pat. No. 4,715,367 discloses a behavioral modification device which may be used to detect and treat snoring, bruxism, and sleep apnea. The patent discloses the use of pressure sensors mounted on either side of the forehead and actuated by flexing the temporal muscles. The patent also discloses using microphones to sense breathing and snoring. The output of the system is a regulatable electric shock applied to the user through a neck collar.

U.S. Pat. No. 6,093,158 discloses a system for monitoring an undesired behavioral disorder such as bruxism, jaw clenching, or snoring. A processor correlates the monitored behavior with the onset of the undesired disorder. Typically the warning device causes the patient to experience an unpleasant sensation, thus promoting the discontinuance of the behavior. The system may record the monitored data related to the undesired behavioral disorders. This feature allows the patient to receive data related to the rate, duration, intensity, and time of day that the unconscious behavior occurred thus allowing the patient to correlate the undesired behavior with outside factors.

U.S. Patent Application No. 2013/0144190 A1 discloses a method for assessing sleep in a normal sleep environment by detecting audible sounds from the patient in a normal sleep environment using a mobile electronic device having a sound sensor, and by determining whether said audible sounds are indicative of normal sleep or a disorder present in said patient.

Although a variety of different systems have been devised to prevent and/or modify a patient's tendency towards bruxism, these systems have typically met only limited success for a variety of reasons. For example, many systems are unreasonably uncomfortable, making normal sleep or day time use impossible. Therefore an improved method of modifying a patient's behavior, specifically behavior associated with bruxism is desirable.

### SUMMARY

It is an object of the invention to provide a solution for detection of teeth grinding using audio which is recorded by a mobile device. The mobile device preferably comprises a mobile phone, a Personal Digital Assistant (PDA), a tablet computer, or a laptop. The audio that is recorded is analyzed by a computer program using pattern recognition and machine learning. Machine learning is related to systems that can learn from data, rather than follow only explicitly programmed instructions. The computer program may be trained to determine a patient's personal teeth grinding sounds. For this purpose a personal algorithm for each patient to detect teeth grinding sounds may be created. This detection may be quantified in a score. This score may be used to compare the amount of teeth grinding over time. In this way the score may be used to verify if any change of behavior have affected the amount of teeth grinding. In this way the invention enables measuring the effect of behavior modification over time by a mobile device and enables the patient to adjust his behavior and/or lifestyle based on the advice provided by the mobile device. This will ultimately lead to reduction of causes of especially secondary bruxism and consequently to reduction or even complete absence of bruxism (including clenching) of the patient. The positive effect of the invention is expected to be structural and sustainable.

### DESCRIPTION OF DRAWINGS

The figures show views of embodiments in accordance with the present invention.

FIG. 1 shows an exemplary algorithm for a mobile device application for detection, presentation, analysis and computing of levels of teeth grinding.

FIG. 2 shows an exemplary algorithm for a mobile device application entering or changing a user profile and presenting advice by the mobile device based on levels of teeth grinding and user profile.

### DETAILED DESCRIPTION

The invention is now described by the following aspects and embodiments, with reference to the figures.

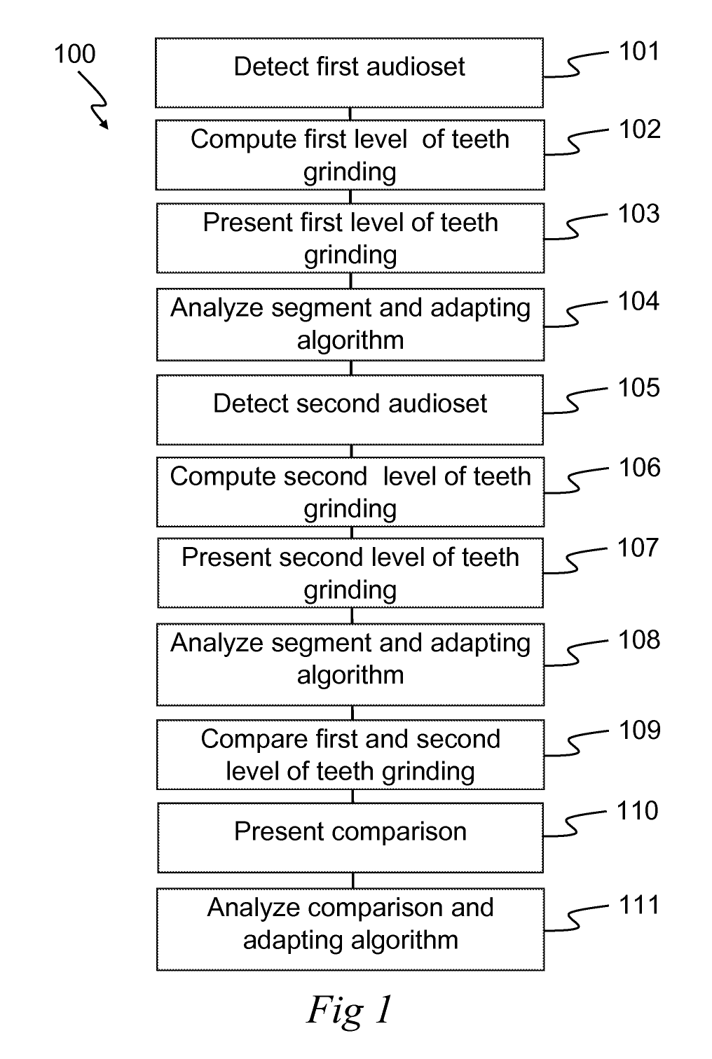

FIG. 1 shows an exemplary algorithm 100 for a mobile device application for detection, presentation, analysis and computing of levels of teeth grinding. The mobile device, by means of a sound sensor such as a built in microphone, detects (which may include recording) 101 a first set of audible sounds (hereinafter referred to as “audioset”) coming from the patient. The mobile device may have a mobile application (hereinafter referred to as “mobile app”) running which facilitates detecting recording, computing and/or analyzing of the audioset. The patient may be asleep when the detection takes place. By employing an algorithm running in software on the mobile device or outside the mobile device, for example in the cloud, a first level of teeth grinding is computed 102 from the first audioset, and it may be established that the patient grinds his teeth. A level of zero or beneath a certain low threshold, may be considered as an indication that the patient does not grind teeth. The level of teeth grinding is recorded and presented 103 to said patient. Preferably this is done by the mobile device via the mobile app. Presentation 103 of the level of teeth grinding may be executed for example the next morning as soon as the patient is awake and consults his mobile app. Alternatively or additionally, the level of teeth grinding may be presented to a physician who is treating the patient. The data in relation to the teeth grinding detection may for that purpose be transmitted by the mobile device through e.g. a wireless telecommunication network to said physician. The computer program (running in the mobile device as part of the mobile app or running outside the mobile device e.g. in the cloud) analyzes 104 a segment of the recorded audioset. Alternatively or additionally the patient and/or the treating physician may adapt 104 the algorithm, in order to decrease the number of false positives in the computing of the current or next level of teeth grinding and/or analysis of the current or next audioset. The process continuous with at least one following iteration of detecting teeth grinding in a similar manner. A second audioset is detected 105 and a second level of teeth grinding is computed 106. The second level of teeth grinding is presented 107. Patient, physician and/or the computer program may analyze 108 a segment of the second audioset and decide to adapt the algorithm. Because there are now two levels of teeth grinding detected, computed and presented, the first and the second level of teeth grinding may be compared 109. The results of the comparison may be presented 110 to patient, physician and/or the computer program, and the comparison is analyzed 111 and the algorithm is adapted 111 if appropriate.

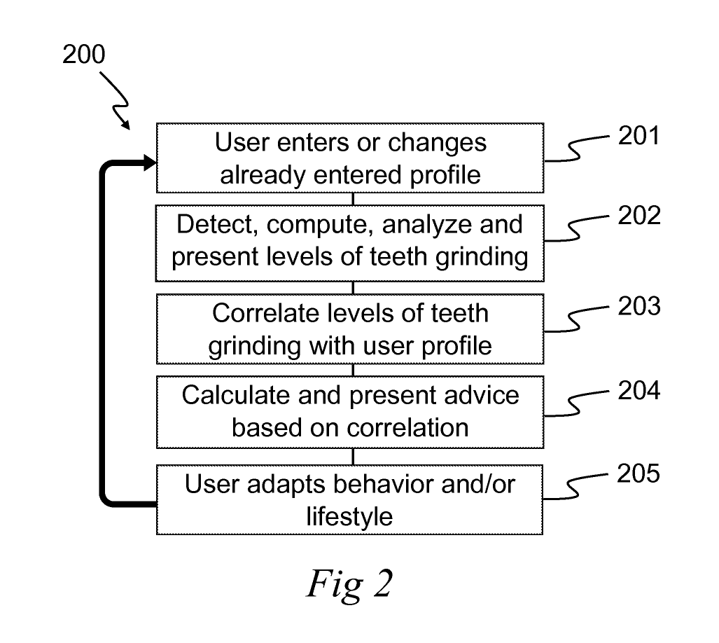

FIG. 2 shows an exemplary algorithm 200 for a mobile device application of entering or changing a user profile and presenting advice by the mobile device based on levels of teeth grinding and user profile. This procedure may be part of the adapting steps 104, 108 and/or 111 of FIG. 1. The step of entering 201 a profile in the mobile device may precede the process of detecting and so forth. Subsequently the audio set is detected, computed, analyzed and presented 202 as described above. In this case, however, the levels of teeth grinding are alternatively or additionally correlated 203 with the user profile. Based on this correlation a calculation is made by the patient, the physician and/or the computer program which leads to an advice presented to the patient 204. This advice may be an advice to change particular behavior of the patient, based on the user profile. For example a patient who drinks a lot of alcohol before going to sleep may be advised for example to reduce the intake of alcohol. It may also be a therapeutic advice directed to a general personal development, or the advice may in particular be related to reduction of stress. Consequently the patient may adapt 205 his behavior and/or lifestyle. This may lead to a changing of the user profile 201. The following steps may be taken in iteration until the desired level of teeth grinding, optimally being zero, is reached.

In a first aspect of the present invention a method for detecting teeth grinding of a patient is proposed, said method comprising the step of detecting a first set of audible sounds from the patient using a mobile device having a sound sensor and a computer program running on said mobile device, wherein said method further comprises the step of computing, using said first set of audible sounds as input for an algorithm, a first level of teeth grinding of said patient.

The exemplary embodiments of the first aspect are that the method further comprises the steps of:
Presenting by the mobile device to said patient the first level of teeth grinding.
Analyzing at least a segment of said first set of audible sounds and adapting said algorithm based on the analysis of said segment.
Detecting a second set of audible sounds from the patient using said mobile device.
Computing, using said second set of audible sounds as input for said adapted algorithm, a second level of teeth grinding of said patient.
Presenting by said mobile device to said patient the second level of teeth grinding.
Analyzing at least a segment of said second set of audible sounds and adapting said algorithm based on the analysis of said segment.
Computing a comparison between said first level of teeth grinding and said second level of teeth grinding.
Presenting by said mobile device to said patient said comparison.
Analyzing said comparison and adapting said algorithm based on the analysis of said comparison. Said analyzing steps and/or said adapting steps may be performed manually, or by said computer program using pattern recognition and/or machine learning.

said computing steps may comprise assessing characteristics of said sounds, said characteristics comprising any one of the group of characteristics comprising:
the amplitude of said sounds;
the interval between said sounds;
the frequency composition of said sounds;
the duration of said sounds,
   and wherein said computing step comprise determining the first and/or second level of teeth grinding as a result of at least one of said characteristics. Said computing steps may comprise using waveform autocorrelation, frequency analysis for identifying an increased variation and power spectrum shift towards higher frequencies, an analysis of cepstral coefficients, or a hidden Markov model.

The inventive method may further comprise:
Recording said audible sounds on a first memory unit comprised in said mobile device.
Transmitting said audible sounds with said mobile device to a computer.
Recording said transmitted audible sounds on a first memory unit comprised in said computer.
Obtaining a Fourier transform and/or a Discrete Fourier transform of said segment of said first set of audible sounds and/or said segment of said second set of audible sounds.
Detecting said audible sounds in stereo using said sound sensor and a second sound sensor.
Detecting video signals from said patient.
Using one or more sensors to measure oxygen saturation, breathing, heart rate, electrocardiographic information, posture, body movements, electroencephalographic information, nasal air flow, oral air flow, CO2 levels, body temperature, air temperature, and/or bioelectrical impedance.

The method may further comprise the steps of configuring a personal profile of the patient on said mobile device and using the personal profile as input for further determining by said computer program the first level of teeth grinding of the patient. Said configuring step may comprise that the patient enters personal data in the mobile device through an interface of the mobile device and said data are stored on the first and/or a second memory unit comprised in the mobile device or on the first and/or a second memory unit comprised in said computer. Said personal data may comprise data of the group comprising:
Gender
Age
Behavioral pathogen, such as smoking, drinking or drugs abuse
A level of feeling rested after sleeping
Weight
Body measurements such as length
Diagnosed disorders such as teeth grinding, snoring, sleep apnea
Use of medicines
Medical Constitution
Condition
A level of well-being

The method may further comprise the step of the computing device presenting an advice for adjusting behavior and/or lifestyle of the patient based on calculation by the computer program of correlations between the personal profile in relation and the computed first level of teeth grinding, the second level of teeth grinding and/or the comparison of the first level and the second level of teeth grinding of the patient.

Said computer program may comprise a mobile app and said algorithm may be executed in any computer of the group of computers comprising:
An external computer operative in the cloud and accessible through Internet;
An external computer in a local area network;
The mobile device.

The term “substantially” herein, such as in “substantially . . . ” etc., will be understood by the person skilled in the art. In embodiments the adjective substantially may be removed. Where applicable, the term “substantially” may also include embodiments with “entirely”, “completely”, “all”, etc. Where applicable, the term “substantially” may also relate to 90% or higher, such as 95% or higher, especially 99% or higher, including 100%. The term “comprise” includes also embodiments wherein the term “comprises” means “consists of”.

It should be noted that the above-mentioned embodiments illustrate rather than limit the invention, and that those skilled in the art will be able to design many alternative embodiments without departing from the scope of the appended claims. In the claims, any reference signs placed between parentheses shall not be construed as limiting the claim. Use of the verb “to comprise” and its conjugations does not exclude the presence of elements or steps other than those stated in a claim. The term “and/or” includes any and all combinations of one or more of the associated listed items. The article “a” or “an” preceding an element does not exclude the presence of a plurality of such elements. The article “the” preceding an element does not exclude the presence of a plurality of such elements. In the device claim enumerating several means, several of these means may be embodied by one and the same item of hardware. The mere fact that certain measures are recited in mutually different dependent claims does not indicate that a combination of these measures cannot be used to advantage.

1. A method for detecting teeth grinding of a patient, said method comprising the step of detecting a first set of audible sounds from the patient using a mobile device having a sound sensor and a computer program running on said mobile device, wherein said method further comprises the step of computing, using said first set of audible sounds as input for an algorithm, a first level of teeth grinding of said patient.

2. The method of claim 1, wherein said method comprises presenting by said mobile device to said patient the first level of teeth grinding.

3. The method of claim 1, wherein said method further comprises the step of analyzing at least a segment of said first set of audible sounds and adapting said algorithm based on the analysis of said segment.

4. The method of claim 1, wherein the method further comprises the step of detecting a second set of audible sounds from the patient using said mobile device.

5. The method of claim 1, wherein said method further comprises the step of computing, using said second set of audible sounds as input for said adapted algorithm, a second level of teeth grinding of said patient.

6. The method of claim 1, wherein said method comprises presenting by said mobile device to said patient the second level of teeth grinding.

7. The method of claim 1, wherein said method further comprises the step of analyzing at least a segment of said second set of audible sounds and adapting said algorithm based on the analysis of said segment.

8. The method of claim 1, wherein the method further comprises the step of computing a comparison between said first level of teeth grinding and said second level of teeth grinding.

9. The method of claim 1, wherein the method further comprises the step of presenting by said mobile device to said patient said comparison.

10. The method of claim 1, wherein said method further comprises the step of analyzing said comparison and adapting said algorithm based on the analysis of said comparison.

11. The method of claim 1, wherein said method comprises that said analyzing steps and/or said adapting steps are performed manually.

12. The method of claim 1, wherein said method comprises that said analyzing steps and/or said adapting steps are performed by said computer program using pattern recognition and/or machine learning.

13. The method of claim 1, wherein said computing steps comprise assessing characteristics of said sounds, said characteristics comprising any one of the group of characteristics comprising:
the amplitude of said sounds;
the interval between said sounds;
the frequency composition of said sounds;
the duration of said sounds,
and wherein said computing step comprise determining the first and/or second level of teeth grinding as a result of at least one of said characteristics.

14. The method of claim 1, wherein said computing steps comprise using waveform autocorrelation, frequency analysis for identifying an increased variation and power spectrum shift towards higher frequencies, an analysis of cepstral coefficients, or a hidden Markov model.

15. The method of claim 1, wherein said method comprises recording said audible sounds on a first memory unit comprised in said mobile device.

16. The method of claim 1, wherein said method comprises transmitting said audible sounds with said mobile device to a computer.

17. The method of claim 1, wherein said method comprises recording said transmitted audible sounds on a first memory unit comprised in said computer.

18. The method of claim 1, wherein said computing steps comprise obtaining a Fourier transform and/or a Discrete Fourier transform of said segment of said first set of audible sounds and/or said segment of said second set of audible sounds.

19. The method of claim 1, wherein said method comprises detecting said audible sounds in stereo using said sound sensor and a second sound sensor.

20. The method of claim 1, wherein said method comprises detecting video signals from said patient.

21. The method of claim 1, wherein said method comprises using one or more sensors to measure oxygen saturation, breathing, heart rate, electrocardiographic information, posture, body movements, electroencephalographic information, nasal air flow, oral air flow, CO2 levels, body temperature, air temperature, and/or bioelectrical impedance.

22. The method of claim 1, wherein the method further comprises the steps of:
Configuring a personal profile of the patient on said mobile device;
Using the personal profile as input for further determining by said computer program the first level of teeth grinding of the patient.

23. The method of claim 1, wherein said configuring step comprises that the patient enters personal data in the mobile device through an interface of the mobile device and said data are stored on the first and/or a second memory unit comprised in the mobile device or on the first and/or a second memory unit comprised in said computer.

24. The method of claim 1, wherein said personal data comprise data of the group comprising:
Gender
Age
Behavioral pathogen, such as smoking, drinking or drugs abuse
A level of feeling rested after sleeping
Weight
Body measurements such as length
Diagnosed disorders such as teeth grinding, snoring, sleep apnea
Use of medicines
Medical Constitution
Condition
A level of well-being

25. The method of claim 1, wherein the method further comprises the step of the computing device presenting an advice for adjusting behavior and/or lifestyle of the patient based on calculation by the computer program of correlations between the personal profile in relation and the computed first level of teeth grinding, the second level of teeth grinding and/or the comparison of the first level and the second level of teeth grinding of the patient.

26. The method of claim 1, wherein said computer program comprises a mobile app.

27. The method of claim 1, wherein said algorithm is executed in any computer of the group of computers comprising:
An external computer operative in the cloud and accessible through Internet;
An external computer in a local area network;
The mobile device.
